(19) United States
(12) Patent Application Publication
(10) Pub. No.: US 2014/0378877 A1
(43) Pub. Date: Dec. 25, 2014

(54) Apparatus and method for relieving weather related arthritic joint pain

(71) Applicants: Sakshi Agarwal, Pleasanton, CA (US); Samir K. Agarwal, Pleasanton, CA (US)

(72) Inventors: Sakshi Agarwal, Pleasanton, CA (US); Samir K. Agarwal, Pleasanton, CA (US)

(21) Appl. No.: 13/924,600

(22) Filed: Jun. 23, 2013

(51) Int. Cl. A61H 1/00; A61F 7/00

(52) U.S. Cl. CPC A61H 1/008; A61F 7/00
USPC 601/18

(57) ABSTRACT

Apparatus and method for relief of arthritic joint pain aggravated due to change in weather, especially cold, rainy weather. The invention offers a practical method to address this pain while allowing user mobility and without being seen with overt medical contraptions. The invention consists of a specialized pressure sleeve with air at a specified temperature and pressure, design to fit joints and specialized clothing to fasten the sleeves. The method describes the usage of the apparatus to achieve the said goal.

### CROSS-REFERENCE TO RELATED APPLICATIONS

Not Applicable

### BACKGROUND OF THE INVENTION

Arthritic joint pain tends to get aggravated during cold, rainy weather. This weather is typically indicative of low atmospheric pressure in a region. The impact of low pressure on the tissue is that it causes the tissue to expand against the joint and nerves exacerbating arthritic pain.

While there is a substantial body of prior art in the area of bone and joint related innovations, as well as in the area of pressure “wearables”, there is nothing we could find that addresses the area of weather related arthritic pain and the apparatus and method described in this invention.

Prior art has focused on manufacturing smart pressure monitored suits which deal with the use of Lycra, and how the material is plotted and cut. Some prior work in the area of pressure garments has focused on prevention of blood accumulating in one region of the body by offering pressure suite with filled liquid strips. Others have addressed appropriate cutting and knitting patterns for pressure garments to prevent deep vein thrombosis. There are other prior works where pressure suits have been built to address the very low (near zero) pressure situations that are encountered in space or at very high altitudes. The inventions there focus on survival of humans, mobility of humans and the “breathing” aspect of the internal clothing. One work of prior art has focused on use of heat and pressure on arthritic pain through the use of hot air being blown (by device like hair dryer) into a bag, where a person inserts the limb and joint into that bag. This invention does not address specific situations due to weather related arthritic pain, and is very different from what is being suggested in our invention, in that we don't require a constant stream of hot air blowing, which makes practical use of the device improbable if not impossible.

### BRIEF SUMMARY OF THE INVENTION

This invention describes an apparatus and method for relief of arthritic joint pain aggravated due to change in weather, especially cold, rainy weather. This weather is emblematic of low pressure. Tissues in the joint tend to expand and impinge on the nerves making arthritic pain worse. The invention takes a unique approach towards counteracting this low pressure situation. The approach also offers a solution that is practical in day to day use, and does not require the user to be tied into stationary medical devices, or be identified as wearing medical contraptions.

The invention counteracts this low pressure situation with surrounding the joints with a wearable sleeve that is filled with air at controlled temperature and pressure. The wearable sleeve is specifically designed for joint wear and is fitted in specially designed clothing that can hold the sleeve. An external, calibrated pump is used to fill the sleeve with air at selected pressure and temperature. The method describes the configuration and usage of the said apparatus to achieve the said goal.

### BRIEF DESCRIPTION OF THE DRAWINGS

So that the manner in which the above recited features, advantages and objects of the present invention are attained and can be understood, the embodiments thereof are illustrated in the appended drawings.

It is to be noted, however, that the appended drawings illustrate only typical embodiments of this invention, and therefore not to be considered limiting of its scope, for the invention may admit to other equally effective embodiments.

FIG. 1 illustrates the front view of the flattened pressure sleeve, as seen from the outside.

FIG. 2 shows the same pressure sleeve, but as viewed from the other side in FIG. 1. It illustrates how the outer layer is wider and larger than the inner layer. It also shows the split fasteners (in form of Velcro, zippers or others), and a bendable seam for easier joint movement. It also shows the air valve through which air is introduced in the sleeve.

FIG. 3 shows the specialized clothing that holds the pressure sleeve. The example here shows inner lining for a pair of trousers. The figure also shows the front holding loops on the clothing, in specific placement.

FIG. 4 shows a zoomed-in, sectional view of how the pressure sleeve is held by the loop on the clothing.

FIG. 5 shows a cross-sectional view of the fully ready to wear apparatus including the outer clothing (such as trousers, shirts, etc.), the pressure sleeve, and the inner lining.

### DETAILED DESCRIPTION OF THE INVENTION

In order to further explain the principle and the structure of the present invention, preferred embodiments will be described in details with reference to the drawings hereinafter. However, it is understood that the embodiments are only used to describe and explain the present invention, not to limit the scope of the present invention.

The two main parts of the apparatus are the especially configured pressure sleeve for the wearer's joint, and an inner lining (comfort clothing) that is especially configured to provide optimal support to keep the pressure sleeve in place. These two parts work together to provide relief against arthritic pain due to weather conditions. Empirical data shows that arthritis patients frequently suffer enhanced pain in muscles and joints during cold, rainy weather, even while they are indoors in heated dwellings. The best understanding of the phenomenon is attributed to the fact that such weather is an indication of low atmospheric pressure setting in, which causes the tissues to expand. As the tissue pushes against joints and the nerves, it exacerbates the pain.

The invention pertains to creating a pressure ring around the joint that negates the effect of low atmospheric pressure. This is achieved using a pressure sleeve.

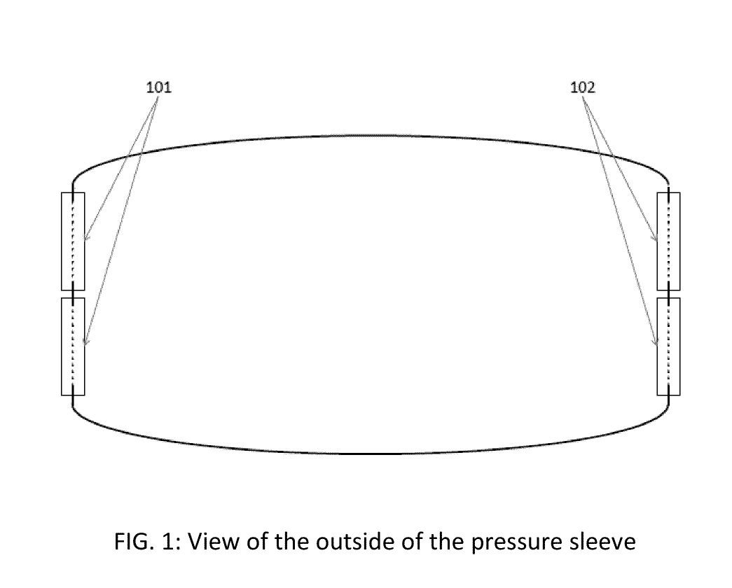

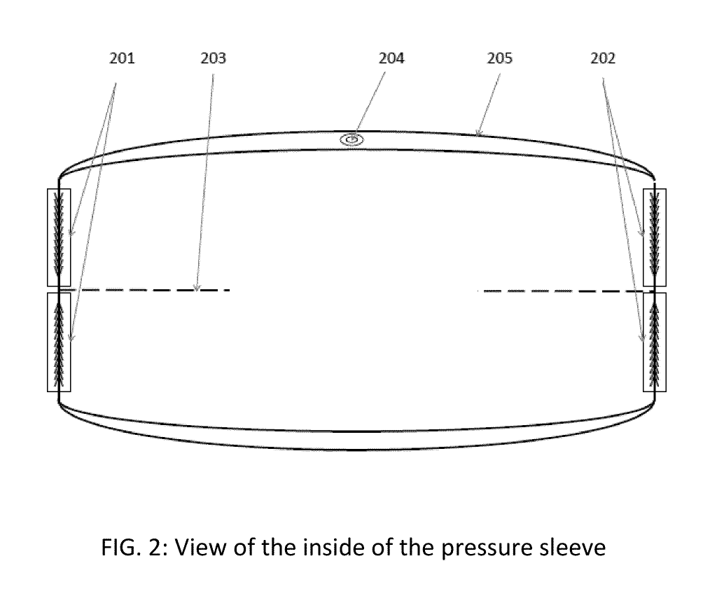

The pressure sleeve is a thin, supple plastic “pouch”, with a larger outer face than an inner one [FIG. 1]. The sleeve rolls around the elbow or knee joint, and is held in place by the fasteners 101, 102. The inner face [FIG. 2] is smaller than the outer face, thus allowing room for the pressure sleeve to bend and stretch as the wearer exercises the joints, without being painful. 201 and 202 show the inside view of the fasteners that close the loop when the pressure sleeve is worn. The fasteners hold the ends of the sleeve firmly in place together, and are realized through Velcro, zippers, or other similar mechanisms. The fasteners have split upper and lower parts to allow easier bending of the pressure sleeve along the joint axis.

203 shows the “joint comfort bendable seam” on the inner face of the pressure sleeve. This seam is staggered towards the ends, so that the ends of the sleeve provide flexibility to bend while the central part does not. When the ends of the pressure sleeve are fastened together, the bendable seam sits at the axis of the joint on the inside of the joint. This seam allows bending the joint comfortably. The central part stays on top of the joint and covers the joint completely.

204 shows the one-way air intake valve for the pressure sleeve. This valve sits at what would be the natural top boundary of the sleeve even though it is continuous material that runs from the outer to the inner face of the pressure sleeve (like a tube). This is where the externally pressure and temperature configured air compressor is connected through an appropriate nozzle. The needle air nozzle, typically used for filling air into basketball and football, is used to fill air at the appropriate pressure and temperature. The method describes the appropriate pressure and temperature settings.

205 shows the wider outer edge of the pressure sleeve, as compared to the inner part of the sleeve. This allows for the stretch encountered by the sleeve when the wearer bends the joint, while still maintaining the pressure around the joint.

The structure of the pressure sleeve is designed to be a seamless tube that slightly tapers towards the ends. This ensures full coverage of the joint, while at the same time providing comfortable under joint coverage. This is also facilitated through the bendable seam that only appears under joint but not on the outside of the joint.

While this pressure sleeve can be directly worn on the joint, the invention further defines specialized clothing apparatus for more comfortable wearing experience. The invention further defines the associated clothing that both holds the pressure sleeve in place, and allows it to be worn comfortably without the plastic touching the skin of the wearer.

The invention describes a clothing system, consisting an inner lining. This lining is specially equipped to hold the pressure sleeve. The outer garment (such as trousers, shirts, etc.) is appropriately fashioned so as to not reveal any special apparatus is being worn. The inner lining provides the comfort factor and support for the pressure sleeve. The invention proposes that the user of the apparatus inserts the similarly shaped inner lining into the outer garment (such as the trousers). The user then inserts the pressure sleeve through a system of loops as described below in the method.

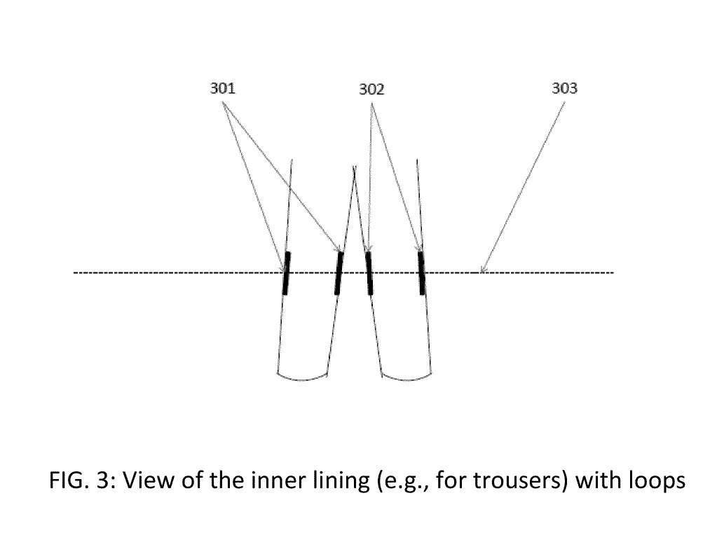

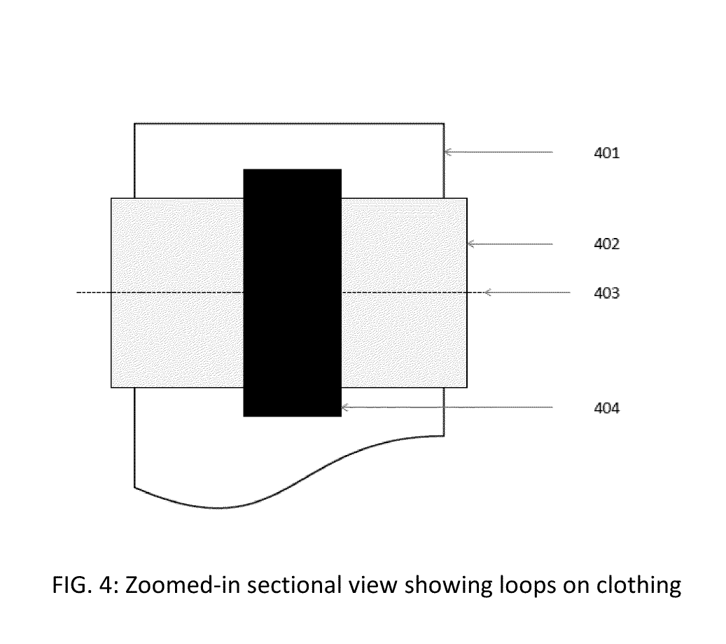

FIG. 3 shows the inner lining 301 and 302 show the sleeve holding loops stitched on to the inner lining that would go with a pair of trousers. The loops are stitched on the outside of this lining. There are a total of four loops for each limb. The loops are arranged in a manner around the lining sleeve so that they are at 90% from each other on the horizontal plane. The loop arrangement is also such that the center of each loop aligns with the axis of the joint of the wearer, as shown in 303. The loops are wide enough and deep enough to allow the pressure sleeve to pass through comfortably but snugly. FIG. 4 illustrates the structure of the loop. 401 shows the lining and how each of the loops is mounted. 402 shows a section of the pressure sleeve fitting snugly through the loop shown as 404. The loop is centered along the axis of joint (403) of the wearer for maximum comfort in exercising the joint.

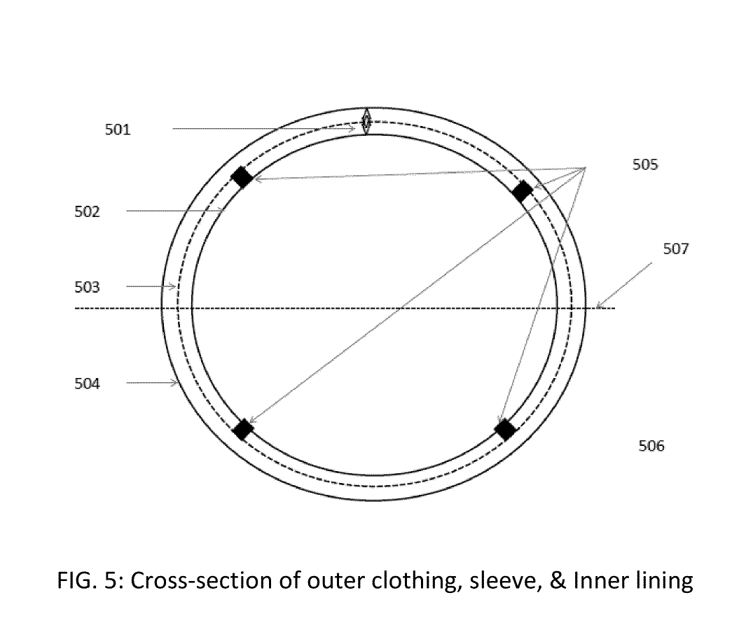

Thus the four loops line up centered at the same axis and at right angles to each other to best hold the pressure sleeve. FIG. 5 illustrates through a cross-section, the full layout of the outer garment, the pressure sleeve, and the inner lining. 507 shows the axis of the joint along which the wearer exercises or bends the joint. 501 shows the split fasteners that hold the pressure sleeve in a ring formation around the joint. 502 shows the inner lining. 503 shows the cross section of the pressure sleeve. 504 shows the section of the outer garment. 505 show the diagonally opposite sleeve loops on the outside of the inner lining. 506 show the diagonally opposite sleeve loops on the inside of the outer garment.

The apparatus is most effectively utilized through the method of use described herein.

A specialized external compressor is used that measures the current atmospheric pressure. It then computes the difference between 765 mm of Hg pressure (14.79 psi or 102 kPa) and the ambient atmospheric pressure. The goal is to maintain a pressure on joint just above the atmospheric pressure at sea level. The pressure sleeve is inflated to this difference in pressure using the pump. If the atmospheric pressure is higher than 765 mm of Hg then the pump does not operate. The air is warmed to 99 degrees Fahrenheit by the pump, if the ambient temperature is below 99 degrees. Finally, the pump is connected to the pressure sleeve using a needle nozzle, and the sleeve is filled with air to the above targeted temperature and pressure.

The pressure sleeve is then ready for use. If worn as part of the clothing described in the invention, the following steps should be followed to fit the pressure sleeve. Fill the pressure sleeve as described above. Insert the sleeve through the four holding loops on the outside of the inner lining, in such a way that the outer part of the pressure sleeve faces outwards over the joint, and the bendable seam faces outward at the bend of the wearer's joint. Then wear this lining and align the pressure sleeve with the joint for most coverage on the joint outside, and aligning the bend in the sleeve with the axis of joint. Next join the split fasteners on the pressure sleeve so a snug pressure ring is created around joint. The user may then wear normal outside clothing over this apparatus.

A needle nozzle can be used to let air out of the pressure sleeve for removal and stowage.

### PATENT CITATIONS

| | | Publication | | |
|---|---|---|---|---|
| Cited Patent | Filing Date | Date | Applicant | Title |
| U.S. Pat. No. 6,488,645 | Apr. 23, 1998 | Dec. 3, 2002 | Prospective Concepts Ag | Suit for problems |
| | | | | associated with |
| | | | | orthostasis |
| U.S. Pat. No. 6,742,193 | Apr. 27, 2001 | Jun. 1, 2004 | Ilc Dover, Inc. | Environmental pressure |
| | | | | suits and method of |
| | | | | manufacture |
| U.S. Pat. No. 8,386,065 | Dec. 22, 2008 | Feb. 26, 2013 | The Hong Kong | Method for |
| | | | Polytechnic University | manufacturing smart |
| | | | | pressure monitored suits |
| U.S. Pat. No. 7,043,329 | May 6, 2004 | May 9, 2006 | The University Of | Pressure garment |
| | | | Manchester | |
| U.S. Pat. No. 5,003,630 | Oct. 23, 1989 | Apr. 2, 1991 | David Clark Company | Pressure garment |
| | | | Incorporated | |
| U.S. Pat. No. 3,751,727 | Aug. 5, 1968 | Aug. 1, 1973 | NASA | Space suit |
| U.S. Pat. No. 8,257,282 | Dec. 19, 2007 | Sep. 4, 2012 | General Patent, LLC | Pressure pulse/shock |
| | | | | wave apparatus for |
| | | | | generating waves |
| | | | | having plane, nearly |
| | | | | plane, convergent off |
| | | | | target or divergent |
| | | | | characteristics |
| U.S. Pat. No. 7,169,119 | Apr. 25, 2005 | Jan. 30, 2007 | Chan King H, Chan Mei | Arthritic hand or foot |
| | | | H, Chan Ming H,Chan | treatment apparatus |
| | | | Yung C | |

We claim:

1. An apparatus for relieving cold, wet weather aggravated arthritic pain. The said apparatus consists of:
1. Synthetic (plastic) airtight pressure sleeves with joint comfort seam, that can be filled with air at differential pressure and temperature levels—as a mechanism to counteract low pressure on joint and limbs,
2. Design of the aforesaid sleeve to allow movement on hinge joints such as elbow and knee,
3. Design of clothing to hold the aforementioned sleeve for elbow and knee joints
Method for filling the pressure sleeve with air at optimal temperature and pressure, and then setting up with the specialized clothing for use
